(19) United States
(12) Patent Application Publication
(10) Pub. No.: US 2019/0105101 A1
(43) Pub. Date: Apr. 11, 2019

(54) MEDICAL TREATMENT DEVICE, OPERATION METHOD OF MEDICAL TREATMENT DEVICE, AND TREATMENT METHOD

(71) Applicant: OLYMPUS CORPORATION, Tokyo (JP)

(72) Inventor: Masato NARISAWA, Tokyo (JP)

(73) Assignee: OLYMPUS CORPORATION, Tokyo (JP)

(21) Appl. No.: 16/214,223

(22) Filed: Dec. 10, 2018

Related U.S. Application Data
(63) Continuation of application No. PCT/JP2016/069951, filed on Jul. 5, 2016.

(51) Int. Cl. A61B 18/14; A61B 18/08

(52) U.S. Cl. CPC A61B 18/1445; A61B 2017/320094; A61B 18/085

(57) ABSTRACT

A medical treatment device includes a pair of holding members with which a subject part to be joined in a body tissue is clampable; an energy application unit provided on at least one of holding members of the pair of holding members, so as to contact the subject part clamped by the pair of holding members, thereby to apply energy to the subject part; and a processor comprising hardware, the processor being configured to control the energy to be applied from the energy application unit, wherein, in a period from start to end of application of energy to the subject part, the processor is further configured to cause the energy application unit to: apply at least high-frequency energy in a first period; apply only ultrasonic energy in a second period after the first period; and apply at least thermal energy in a third period after the second period.

### CROSS-REFERENCE TO RELATED APPLICATION

This application is a continuation of PCT International Application No. PCT/JP2016/069951 filed on Jul. 5, 2016, the entire content of which are incorporated herein by reference.

### BACKGROUND

The present disclosure relates to a medical treatment device, an operation method of a medical treatment device, and a treatment method.

In recent years, development of medical treatment devices to join parts subject to joining (referred to as subject parts below) in a body tissue by applying energy to the subject parts has been accelerated. While such a medical treatment device does not leave any physical object, such as staples, in a living body and thus is advantageous in less adverse effects on a human body, the strength of joining is lower than that by the staples and the like, and some subject parts cannot be joined depending on the thickness, and therefore there has been a demand for increase in the strength of joining.

The extracellular matrix of a body tissue (such as collagen and elastin) is formed of fibrous texture. Thus, it is assumed that, when subject parts are joined, extracting the extracellular matrix from the subject parts and causing the extracellular matrix to tangle tightly will increase the strength of joining.

The extracellular matrix is thus has been focused on and medical treatment devices for the purpose of increasing the strength of joining have been proposed (for example, refer to Japanese Laid-open Patent Publication No. 2012-239899).

The medical treatment device described in the above Patent Publication clamps subject parts with a pair of jaws and applies mechanical oscillations to the subject parts via the pair of jaws (apply ultrasonic energy to the subject parts), thereby strengthening extraction and mixture of the extracellular matrix.

### SUMMARY

The present disclosure has been made in view of the above and is directed to a medical treatment device, an operation method of a medical treatment device, and a treatment method.

According to a first aspect of the present disclosure, a medical treatment device is provided which a pair of holding members with which a subject part to be joined in a body tissue is clampable; an energy application unit provided on at least one of holding members of the pair of holding members, so as to contact the subject part when the subject part is clamped by the pair of holding members, thereby to apply energy to the subject part; and a processor comprising hardware, the processor being configured to control the energy to be applied from the energy application unit to the subject part, thereby to treat the subject part, wherein in a period from when application of energy to the subject part is started until when treating the subject part is completed, the processor is further configured to cause the energy application unit to: apply at least high-frequency energy to the subject part in a first period; apply only ultrasonic energy to the subject part in a second period after the first period; and apply at least thermal energy to the subject part in a third period after the second period.

According to a second aspect of the present disclosure, an operation method of a medical treatment device is provided. The method includes applying, after subject part to be joined in a body tissue is clamped by a pair of holding members, at least high-frequency energy from at least one of holding members of the pair of holding members to the subject part in a first period, applying only ultrasonic energy from at least one of holding members of the pair of holding members to the subject part in a second period after the first period, and applying at least thermal energy from at least one of holding members of the pair of holding members to the subject part in a third period after the second period.

According to a second aspect of the present disclosure, a treatment method is provided which includes clamping a subject part to be joined in a body tissue with a pair of holding members; applying at least high-frequency energy from at least one of holding members of the pair of holding members to the subject part in a first period, applying only ultrasonic energy from at least one of holding members of the pair of holding members to the subject part in a second period after the first period, and applying at least thermal energy from at least one of holding members of the pair of holding members to the subject part in a third period after the second period.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a medical treatment device according to a first embodiment of the present disclosure;

FIG. 2 is a block diagram illustrating a configuration of a control device illustrated in FIG. 1;

FIG. 3 is a flowchart illustrating joining control performed by the control device illustrated in FIG. 2;

FIG. 4 is a diagram illustrating behavior of impedance of subject parts that is calculated at and after step S4 represented in FIG. 3;

FIG. 5 is a diagram illustrating behavior of impedance of an ultrasonic transducer that is calculated at and after step S8 represented in FIG. 3;

FIG. 6 is a time chart representing types of energy to be applied to the subject parts and compressive loads to be applied to the subject parts in first to third periods in the joining control illustrated in FIG. 3;

FIG. 7 is a diagram illustrating a modification of the first embodiment of the present disclosure;

FIG. 8 is a block diagram illustrating a configuration of a medical treatment device according to a second embodiment of the present disclosure;

FIG. 9 is a diagram illustrating functions of a lock mechanism represented in FIG. 8;

FIG. 10 is a flowchart illustrating joining control performed by the control device illustrated in FIG. 8;

FIG. 11 is a time chart representing types of energy to be applied to subject parts and compressive loads to be applied to subject parts in first to third periods in joining control according to a third embodiment of the present disclosure;

FIG. 12 is a time chart representing types of energy to be applied to subject parts and compressive loads to be applied to the subject parts in first to third periods in joining control according to a fourth embodiment of the present disclosure;

FIG. 13 is a time chart representing types of energy to be applied to subjects part and compressive loads to be applied to the subject parts in first to third periods in joining control according to a fifth embodiment of the present disclosure;

FIG. 14 is a time chart representing types of energy to be applied to subject parts and compressive loads to be applied to the subject parts in first to third periods in joining control according to a sixth embodiment of the present disclosure; and

FIG. 15 is a time chart representing types of energy to be applied to subject parts and compressive loads to be applied to the subject parts in first to third periods in joining control according to a seventh embodiment of the present disclosure.

### DETAILED DESCRIPTION OF THE PREFERRED EMBODIMENTS

Modes for carrying out the present disclosure (referred to as embodiments below) will be described with reference to the drawings. The embodiments to be described below do not limit the disclosure. Furthermore, in description of drawings, the same components are denoted with the same reference numbers.

### First Embodiment

Schematic Configuration of Medical Treatment Device

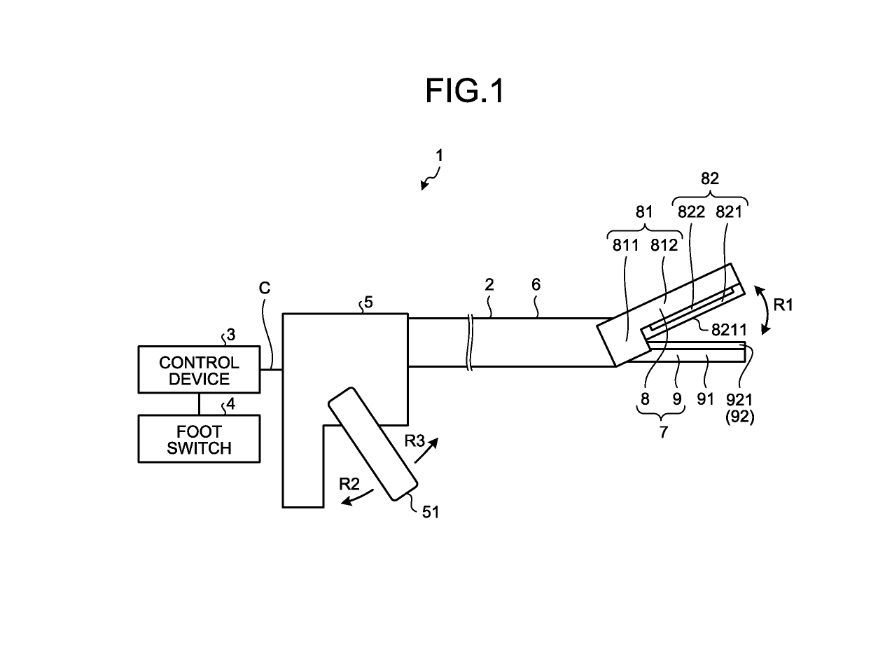

FIG. 1 is a diagram schematically illustrating a medical treatment device 1 according to the first embodiment of the present disclosure.

The medical treatment device 1 applies energy (high-frequency energy, ultrasonic energy and thermal energy) to parts on which treatment (joining or inosculation) is to be performed (subject parts, blow) in a body tissue to treat the subject parts. As illustrated in FIG. 1, the medical treatment device 1 includes a treatment tool 2, a control device 3 and a foot switch 4.

Configuration of Treatment Tool

The treatment tool 2 is a liner-type surgical-medical treatment tool for treating subject parts through the abdominal wall. As illustrated in FIG. 1, the treatment tool 2 includes a handle 5, a shaft 6 and a clamper 7.

The handle 5 is a part that is held by an operator. As illustrated in FIG. 1, the handle 5 is provided with an operation knob 51.

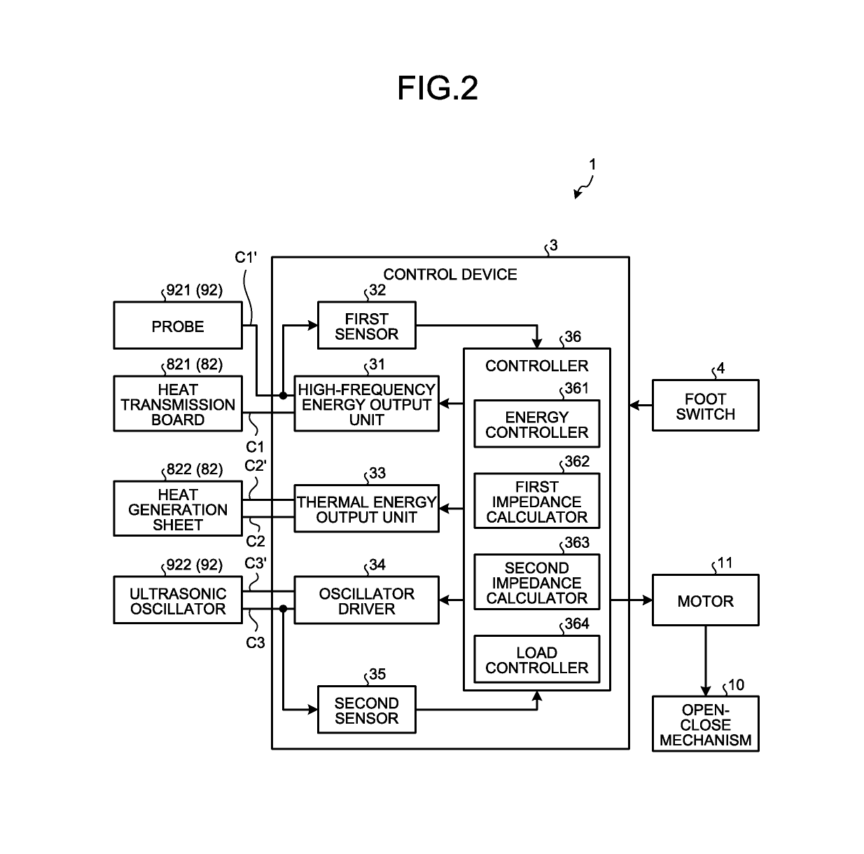

The shaft 6 has an approximately cylindrical shape and has an end that is connected to the handle 5 (FIG. 1). The clamper 7 is connected to the other end of the shaft 6. Inside the shaft 6, an open-close mechanism 10 (see FIG. 2) is provided which causes first and second holding members 8 and 9 (FIG. 1) that form the clamper 7 to open and close according to an operation performed on the operation knob 51 by the operator. Inside the handle 5, a motor 11 (see FIG. 2) is provided which is connected to the open-close mechanism 10 and that, when the subject parts are clamped by the first and second holding members 8 and 9, increases a compressive load to be applied to the subject parts from the first and second holding members 8 and 9 by causing the open-close mechanism 10 to act under the control of the control device 3. Furthermore, inside the shaft 6, an electric cable C (FIG. 1) that is connected to the control device 3 is provided from one end to the other end via the handle 5.

Configuration of Clamper

The clamper 7 is a part that clamps the subject parts and treats the subject parts. As illustrated in FIG. 1, the clamper 7 includes the first holding member 8 and the second holding member 9.

The first and second holding members 8 and 9 are configured as openable and closable in directions of an arrow R1 (FIG. 1) according to an operation performed on the operation knob 51 by the operator (capable of clamping the subject parts).

Specifically, the first holding member 8 is pivotably supported on the other end of the shaft 6. On the other hand, the second holding member 9 is fixed to the other end of the shaft 6. In other words, in the first embodiment, the first holding member 8 is configured as openable and closable with respect to the second holding member 9 according to an operation performed on the operation knob 51 by the operator. For example, when the operation knob 51 moves in the direction of an arrow R2 (FIG. 1), the first holding member 8 rotates in a direction in which the first holding member 8 gets close to the second holding member 9. When the operation knob 51 moves in the direction of an arrow R3 (FIG. 1) in an inverse direction to the arrow R2, the first holding member 8 rotates in a direction in which the first holding member 8 gets apart from the second holding member 9.

The first holding member 8 is arranged on an upper side with respect to the second holding member 9 in FIG. 1. The first holding member 8 includes a first jaw 81 and a first energy application unit 82.

As illustrated in FIG. 1, the first jaw 81 includes a pivotally-supported member 811 that is pivotally supported on the other end of the shaft 6, and a supporting board 812 that is connected to the pivotally-supported member 811, and the first jaw 81 opens and closes in the directions of the arrow R1 according to an operation performed on the operation knob 51 by the operator.

The first energy application unit 82 applies high-frequency energy and thermal energy to the subject parts under the control of the control device 3. As illustrated in FIG. 1, the first energy application unit 82 includes a heat transmission board 821 and a heat generation sheet 822, and the heat generation sheet 822 and the heat transmission board 821 are laminated in this order on a board surface opposed to the second holding member 9 in the supporting board 812.

The heat transmission board 821 is formed of, for example, a thin copper plate.

In the heat transmission board 821, in FIG. 1, when the first and second holding members 8 and 9 clamp the subject parts, the board surface on a lower side functions as a treatment surface 8211 that contacts the subject parts.

The heat transmission board 821 transmits heat from the heat generation sheet 822 to the subject parts from the treatment surface 8211 (applies thermal energy to the subject parts). Furthermore, the heat transmission board 821 has a high-frequency wire lead C1 (see FIG. 2), which forms the electric cable C, connected thereto, and applies high-frequency energy to the subject parts by high-frequency power supplied between the heat transmission board 821 and a probe 921, which will be described below, via the high-frequency leads C1 and C1′ (see FIG. 2) by the control device 3. In other words, the heat transmission board 821 also functions as a high-frequency electrode.

The heat generation sheet 822 is, for example, a sheet heater and functions as a heat generator. Although specific illustration is omitted in the drawings, the heat generation sheet 822 has a configuration in which a resistance pattern is formed by evaporation, or the like, on a sheet substrate formed of an insulating material, such as polyimide.

The resistance pattern is formed along a U shape that follows an outer peripheral shape of the heat generation sheet 822, and has heat generation wire leads C2 and C2′ (see FIG. 2), which forms an electric cable C, connected to corresponding ends of the resistance pattern. Then, the resistance pattern generates heat by being applied with voltage (energized) via the heat generation wire leads C2 and C2′ by the control device 3.

Although illustration is omitted in FIG. 1, an adhesion sheet for adhering the heat transmission board 821 and the heat generation sheet 822 together is interposed between the heat transmission board 821 and the heat generation sheet 822. The adhesion sheet is a sheet that has a high thermal conductivity, is high-temperature resistant and has adhesiveness and is, for example, formed by mixing ceramic with a high thermal conductivity, such as alumina or aluminum nitride, into epoxy resin.

As illustrated in FIG. 1, the second holing member 9 includes a second jaw 91 and a second energy application unit 92.

The second jaw 91 is fixed to the other end of the shaft 6 and has a shape as extending along the axial direction of the shaft 6.

The second energy application unit 92 applies ultrasonic energy to the subject parts under the control of the control device 3. The second energy application unit 92 includes the probe 921 (FIG. 1) and an ultrasonic transducer 922 (see FIG. 2).

The probe 921 is a columnar member that is formed of a conductive material and extends along the axial direction of the shaft 6. As illustrated in FIG. 1, the probe 921 is inserted into the shaft 6 with its one end (right-end side in FIG. 1) exposed to the outside and with the other end to which the ultrasonic transducer 922 is being attached. The probe 921 contacts the subject parts when the first and second holding members 8 and 9 clamp the subject parts and transmits the ultrasonic oscillations that are generated by the ultrasonic transducer 922 to the subject parts (applies ultrasonic energy to the subject parts). The probe 921 has a high-frequency wire lead C1′ (see FIG. 2), which forms the electric cable C, connected thereto, and applies high-frequency energy to the subject parts by high-frequency power applied between the probe 921 and the heat transmission board 821 by the control device 3 via the high-frequency leads C1 and C1′.

The ultrasonic transducer 922 is formed by a piezoelectric oscillator using a piezoelectric element that stretches and shrinks according to application of an alternating current. The ultrasonic transducer 922 has ultrasonic wire leads C3 and C3′ (see FIG. 2), which form the electric cable C, connected thereto, and generates ultrasonic oscillations by being applied with an alternating current under the control of the control device 3.

Although specific illustration is omitted in the drawings, an oscillation enhancing member, such as a horn, that enhances oscillations that are generated by the ultrasonic transducer 922 is interposed between the ultrasonic transducer 922 and the probe 921.

As a configuration of the second energy application unit 92, a configuration to cause the probe 921 to oscillate vertically (oscillation in the axial direction of the probe 921) may be used or a configuration to cause the probe 921 to oscillate horizontally (oscillation in the radial direction of the probe 921) may be used.

Configuration of Control Device and Foot Switch

FIG. 2 is a block diagram illustrating a configuration of the control device 3.

FIG. 2 mainly illustrates a relevant part of the disclosure as the configuration of the control device 3.

The foot switch 4 is a part that the operator operates by foot and, in response to the operation (ON), the foot switch 4 outputs an operation signal to the control device 3. The control device 3 starts joining control, which will be described below, according to the operation signal.

Note that the unit that starts the joining control is not limited to the foot switch 4 and alternatively a switch that is operated by hand may be used.

The control device 3 overall controls operations of the treatment tool 2. As illustrated in FIG. 2, the control device 3 includes a high-frequency energy output unit 31, a first sensor 32, a thermal energy output unit 33, a transducer driver 34, a second sensor 35 and a controller 36.

The high-frequency energy output unit 31 supplies high-frequency power between the heat transmission board 821 and the probe 921 via the high-frequency leads C1 and C1′ under the control of the controller 36.

The first sensor 32 detects a voltage value and a current value that are supplied from the high-frequency energy output unit 31 to the heat transmission board 821 and the probe 921. The first sensor 32 then outputs signals corresponding to the voltage value and the current value that have been detected to the controller 36.

The thermal energy output unit 33 applies a voltage to (energizes) the heat generation sheet 822 via the heat generation leads C2 and C2′ under the control of the controller 36.

The transducer driver 34 applies an alternating voltage to the ultrasonic transducer 922 via the ultrasonic leads C3 and C3′ under the control of the controller 36.

The second sensor 35 detects the voltage value and the current value that are applied from the transducer driver 34 to the ultrasonic transducer 922. The second sensor 35 then outputs signals corresponding to the voltage value and the current value that have been detected to the controller 36.

The controller 36 is configured to include a CPU (Central Processing Unit) or the like and executes the joining control according to a predetermined control program, when the foot switch 4 turns on. As illustrated in FIG. 2, the controller 36 includes an energy controller 361, a first impedance calculator 362, a second impedance calculator 363, and a load controller 364.

The energy controller 361 controls operations of the high-frequency energy output unit 31, the thermal energy output unit 33 and the transducer driver 34 according to an operation signal from the foot switch 4 and each of impedances of the subject parts and the ultrasonic transducer 922 that are calculated respectively by the first and second impedance calculators 362 and 363. In other words, the energy controller 361 controls timing of applying high-frequency energy, ultrasonic energy and thermal energy to the subject parts from the first and second energy application units 82 and 92 to treat the subject parts.

The first impedance calculator 362 calculates the impedance of the subject parts during application of high-frequency energy to the subject parts based on the voltage value and the current value that are detected by the first sensor 32.

The second impedance calculator 363 calculates the impedance of the ultrasonic transducer 922 during application of ultrasonic energy to the subject parts based on the voltage value and the current value that are detected by the second sensor 35.

The load controller 364 causes the motor 11 to operate based on the impedance of the ultrasonic transducer 922, which is calculated by the second impedance calculator 363, and to increase the compressive load that is applied from the first holding members 8 and 9 to the subject parts (power to clamp the subject parts with the first and second holding members 8 and 9).

Operations of Medical Treatment Device

Operations of the above-described medical treatment device 1 will be described.

The joining control performed by the control device 3 will be described mainly as operations of the medical treatment device 1 below.

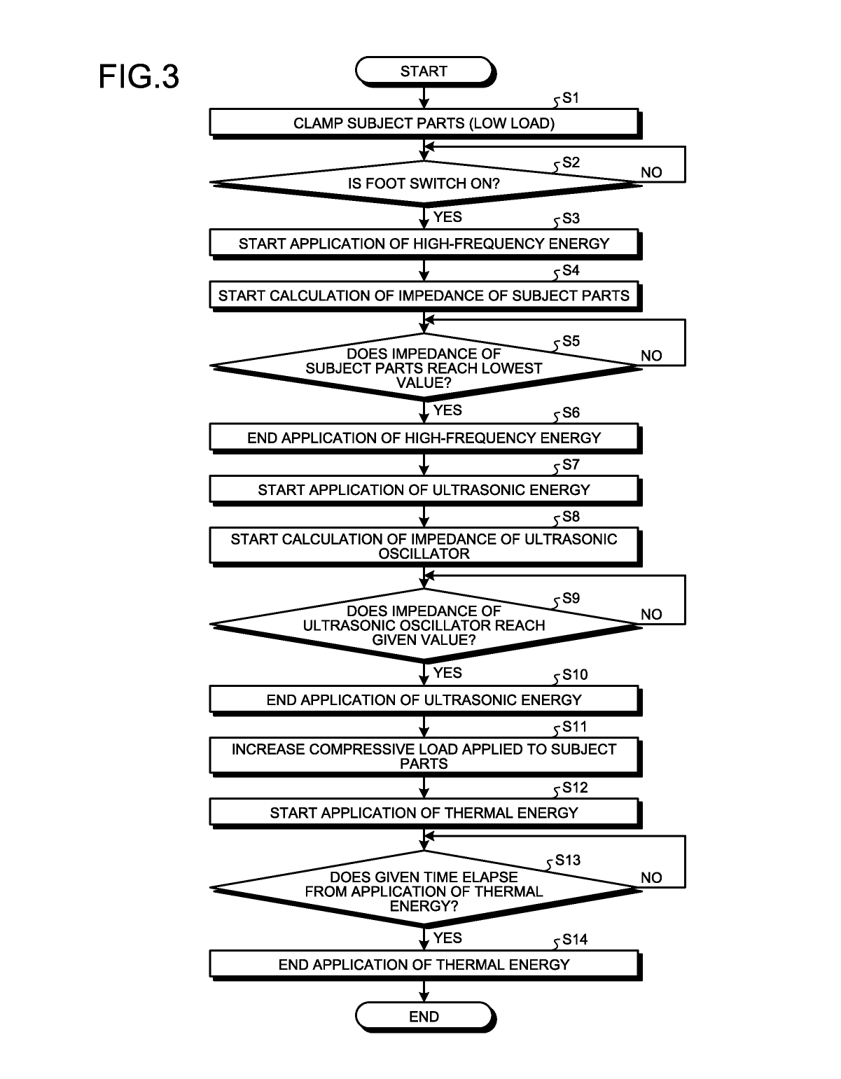

FIG. 3 is a flowchart illustrating the joining control performed by the control device 3.

The operator holds the treatment tool 2 and inserts the tip part (part of the clamper 7 and the shaft 6) of the treatment tool 2 into the abdominal cavity through the abdominal wall with, for example, a trocar, or the like. The operator then operates the operation knob 51 to open and close the first and second holding members 8 and 9 to clamp subject parts with the first and second holding members 8 and 9 (step S1: clamping step).

The operator then operates the foot switch 4 to cause the control device 3 to start the joining control.

When an operation signal from the foot switch 4 is input (the foot switch 4 is on) (step S2: YES), the energy controller 361 drives the high-frequency energy output unit 31 to start supplying high-frequency power from the high-frequency energy output unit 31 to the heat transmission board 821 and the probe 921 (start applying high-frequency energy to the subject parts) (step S3: first application step).

After step S3, the first impedance calculator 362 starts calculating an impedance of the subject part based on the voltage value and the current value that have been detected by the first sensor 32 (step S4).

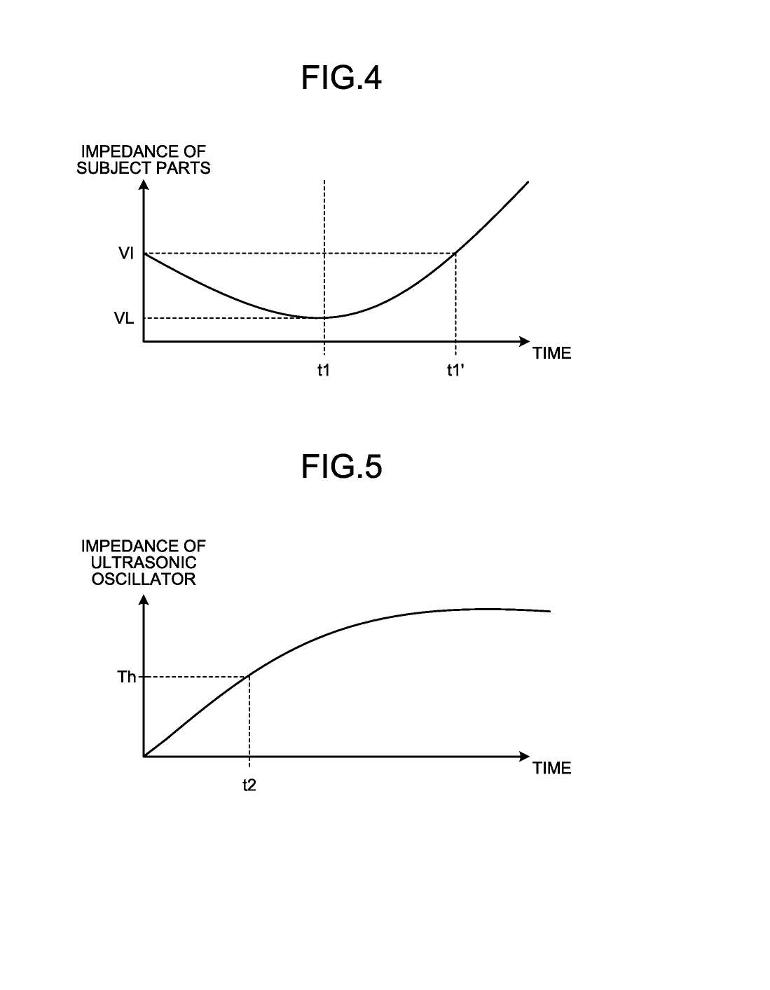

FIG. 4 is a diagram illustrating behavior of impedance of the subject parts that is calculated at and after step S4.

When high-frequency energy is applied to the subject parts, the impedance of the subject parts represents the behavior represented in FIG. 4.

In an initial time period in which high-frequency energy is applied (from start of application of high-frequency energy to Time t1), as illustrated in FIG. 4, the impedance of the subject parts decreases gradually. This results from occurrence of cell membrane destruction of the subject parts because of application of the high-frequency energy and extraction of the extracellular matrix from the subject parts. In other words, the initial time period is a time period in which the extracellular matrix is extracted from the subject parts and the viscosity of the subject parts lowers (the subject parts are softened).

At and after Time t1 when the impedance of the subject part becomes the lowest value VL, the impedance of the subject parts increases gradually as illustrated in FIG. 4. This results from the fact that that Joule heat acts on the subject parts because of application of high-frequency energy and the subject part itself generates heat and thus the moisture of the subject part decreases (evaporates). In other words, a time period at and after Time t1 is a time period in which the extracellular matrix is not extracted from the subject parts and the moisture in the subject parts evaporates due to heat generation and thus the viscosity of the subject part increases (the subject part gets coagulated).

After step S4, the energy controller 361 constantly monitors whether the impedance of the subject parts, which is calculated by the first impedance calculator 362, becomes the lowest value VL (step S5)

When it is determined that the impedance of the subject parts becomes the lowest value VL (step S5: YES), the energy controller 361 stops driving the high-frequency energy output unit 31 (ends applying high-frequency energy to the subject parts) (step S6).

After step S6, the energy controller 361 drives the transducer driver 34 to start application of an alternating voltage from the transducer driver 34 to the ultrasonic transducer 922 (start application of ultrasonic energy to the subject parts) (step S7: second application step).

After step S7, the second impedance calculator 363 starts calculating an impedance of the ultrasonic transducer 922 based on the voltage value and the current value that have been calculated by the second sensor 35 (step S8).

FIG. 5 is a diagram illustrating behavior of the impedance of the ultrasonic transducer 922 that is calculated at and after step S8.

When ultrasonic energy is applied to the subject parts, the impedance of the ultrasonic transducer 922 represents the behavior in FIG. 5.

The impedance of the ultrasonic transducer 922 increases according to the load that is put on the probe 921 when the first and second holding members 8 and 9 are clamping the subject part.

Application of the ultrasonic energy causes the moisture in the subject parts to evaporate and accordingly the viscosity of the subject parts increases. For this reason, the load applied to the probe 921 gradually increases as the subject parts get coagulated at and after Time t1. In other words, the impedance of the ultrasonic transducer 922 increases gradually as illustrated in FIG. 5.

After step S8, the energy controller 361 constantly monitors whether the impedance of the ultrasonic transducer 922 that is calculated by the second impedance calculator 363 becomes a predetermined value Th (FIG. 5) (step S9).

When it is determined that the impedance becomes the predetermined value Th (YES at step S9), the energy controller 361 stops driving the transducer driver 34 (ends application of ultrasonic energy to the subject parts) (step S10).

After step S10, the load controller 364 causes the motor 11 to operate to increase the compressive load that is applied from the first and second holding members 8 and 9 to the subject parts (step S11).

After step S11, the energy controller 361 drives the thermal energy output unit 33 to start application of voltage (energizing) from the thermal energy output unit 33 to the heat generation sheet 822 (start application of thermal energy to the subject parts) (step S12: third application step).

After step S12, the energy controller 361 constantly monitors whether a predetermined time elapses from the application of thermal energy at step S12 (step S13).

When it is determined that the predetermined time elapses (step S13: YES), the energy controller 361 stops driving the thermal energy output unit 33 (ends application of thermal energy to the subject parts) (step S14).

With the above-described joining control, the subject parts have been treated (joined).

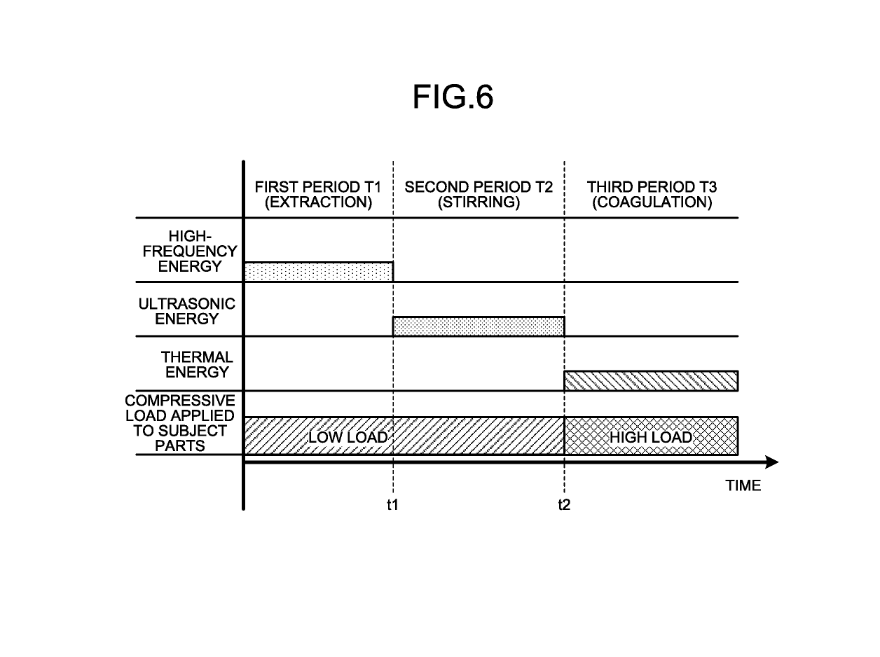

FIG. 6 is a time chart representing types of energy to be applied to the subject part in the first to third Periods T1 to T3 in the joining control represented in FIG. 3 and the compressive load that is applied to the subject part.

Timing of application of each of high-frequency energy, ultrasonic energy and thermal energy, and timing of change of the compressive load applied to the subject parts in the period from start of application of energy to the subject parts to completion of treatment on the subject parts are summarized as represented in FIG. 6.

In other words, in First Period T1 from when the foot switch 4 is turned on until Time t1, as represented in FIG. 6, only high-frequency energy is applied to the subject parts. In First Period T1, the compressive load that is applied to the subject parts from the first and second holding members 8 and 9 is a relatively low load (for example, approximately 0.2 MPa).

In Second Period T2 from Time t1 until Time t2, as represented in FIG. 6, only ultrasonic energy is applied to the subject parts. In Second Period T2, the compressive load to be applied to the subject parts from the first and second holding members 8 and 9 is a load equal to that in First Period T1.

In Third Period T3 from Time t2 until when the predetermined time which is determined at step S13 elapses, only thermal energy is applied to the subject parts. In Third Period T3, the compressive load that is applied to the subject parts from the first and second holding members 8 and 9 is a load higher than those in First and Second Periods T1 and T2.

As described above, in the medical treatment device 1 according to the first embodiment, when the subject part is clamped by the first and second holding members 8 and 9, the compressive load to be applied from the first and second holding members 8 and 9 to the subject parts in Third Period T3 is set higher than those in First and Second Periods T1 and T2.

In other words, increasing the compressive load applied to the subject parts when the extracellular matrix coagulates (in Third Period T3) enables realization of strong joining. Furthermore, lowering the compressive load applied to the subject parts when the extracellular matrix is extracted and stirred (First and Second Periods T1 and T2) may prevent the extracted extracellular matrix from flowing out between the first and second holding members 8 and 9. Furthermore, while, the higher the compressive load applied to the subject parts when the extracellular matrix is stirred is, the more ultrasonic energy (ultrasonic oscillations) is transmitted not to the subject part but to the first jaw 81, keeping the compressive load low as in the first embodiment enables efficient transmission of ultrasonic energy (ultrasonic oscillations) to the subject parts (effectively stirring the extracellular matrix).

After clamping the subject parts with the first and second holding members 8 and 9, the above-explained medical treatment device 1 according to the first embodiment applies, to the subject parts, high-frequency energy in First Period T1, ultrasonic energy in Second Period T2, and thermal energy in Third Period T3. In other words, application of high-frequency energy in First Period T1 causes the cell membrane of the subject parts to be destroyed and to cause the extracellular matrix to be extracted, application of ultrasonic energy in Second Period T2 causes the extracellular matrix to be stirred and to be tightly tangled, and application of thermal energy in Third Period T3 causes the extracellular matrix to coagulate.

Thus, the medical treatment device 1 according to the first embodiment produces an effect that it is possible to properly execute the three processes of extraction, stirring and coagulation of the extracellular matrix that are necessary to join the subject parts and increase the strength of joining between the subject parts.

Incidentally, in order to cause the extracellular matrix to be stirred and to be tightly tangled, the extracellular matrix has to exist flowably between the first and second holding members 8 and 9. When high-frequency energy or thermal energy is applied to the subject parts in Second Period T2 to stir the extracellular matrix, the extracellular matrix is dehydrated because of the effect of heat, such as Joule heating caused by the high-frequency energy, and thus the flowability is lost.

The medical treatment device 1 according to the first embodiment applies only ultrasound energy to the subject parts in Second Period T2 to stir the extracellular matrix. This preferably realize an effect that it is possible to promote three-dimensional tangling of the extracellular matrix while maintaining the flowability of the extracellular matrix and to increase the above-described strength of joining between the subject parts.

The medical treatment device 1 according to the first embodiment starts Second Period T2 when the impedance of the subject parts becomes the lowest value VL.

Accordingly, it is possible to appropriately set First Period T1 in which the extracellular matrix is extracted to execute the stirring process after a sufficient amount of extracellular matrix is extracted from the subject parts, thereby further increasing the strength of joining between the subject parts.

The medical treatment device 1 according to the first embodiment starts Third Period T3 when the impedance of the ultrasonic transducer 922 becomes the predetermined value Th.

Accordingly, it is possible to properly set Second Period T2 in which the extracellular matrix is stirred to execute the coagulation process after stirring the extracellular matrix sufficiently, thereby further increasing the strength of joining between the subject parts.

### Modification of First Embodiment

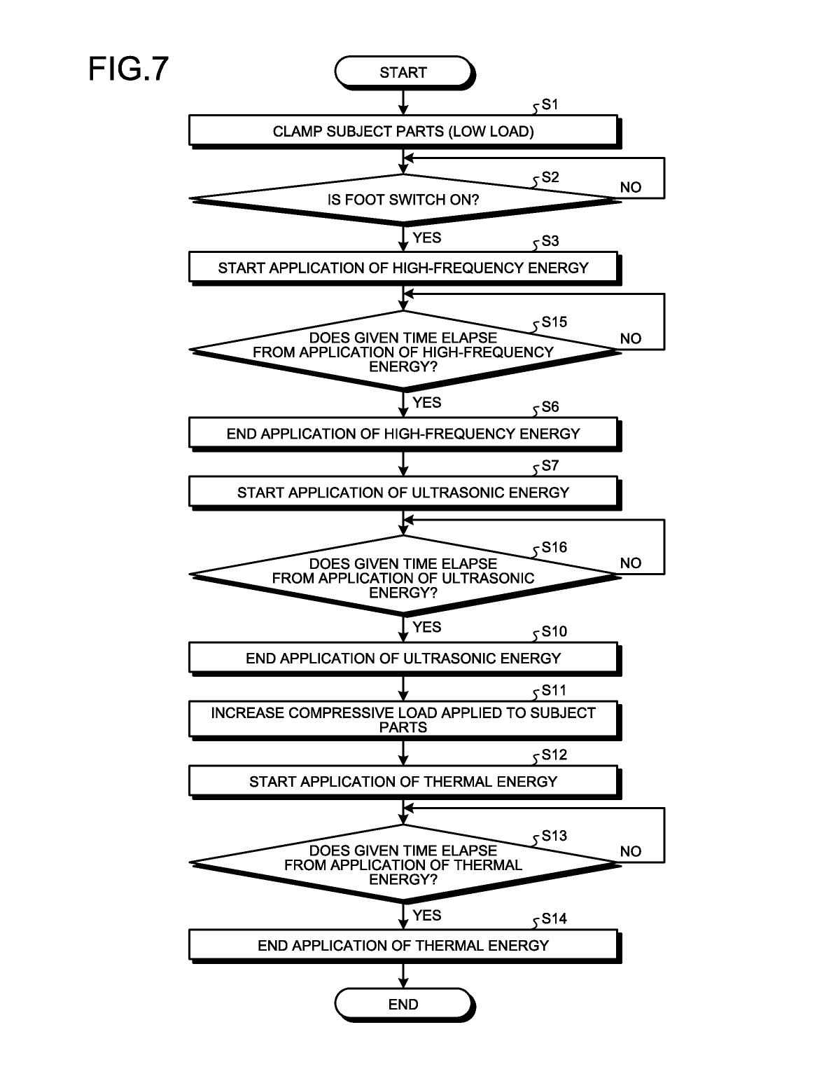

FIG. 7 is a diagram illustrating a modification of the first embodiment of the present disclosure. Specifically, FIG. 7 is a flowchart representing joining control according to the modification.

In the above-described first embodiment, Second Period T2 is started based on the impedance of subject parts and Third Period T3 is started based on the impedance of the ultrasonic transducer 922; however, without limiting to this, a configuration to start Second and Third Periods T2 and T3 when a predetermined time elapses as in this modification may be employed.

In other words, in the modification, the first and second sensors 32 and 35 and the first and second impedance calculators 362 and 363 are omitted. In the joining control in the modification, as illustrated in FIG. 7, steps S4, S5, S8 and S9 relating to calculation of each of impedances of the subject parts and the ultrasonic transducer 922 are omitted and steps S15 and S16 are added.

Step S15 is executed after step S3.

Specifically, at step S15, the energy controller 361 constantly monitors whether a predetermined time elapses from application of high-frequency energy at step S3.

The predetermined time is a time period that is set as follows.

Specifically, each of steps S3 to S5 is executed in advance on plural body tissues. Then, time periods from when application of high-frequency energy is started until when the impedance of the subject parts becomes the lowest value VL are acquired and an average of the acquired times is set as the aforementioned predetermined time on which the determination is made at step S15.

When it is determined that the predetermined time elapses from application of high-frequency energy (step S15: YES), the control device 3 moves to step S6.

Step S16 is executed after step S7.

Specifically, at step S16, the energy controller 361 constantly monitors whether a predetermined time elapses from application of ultrasonic energy at step S7.

The predetermined time is a time period that is set as follows.

In other words, each of steps S3 to S9 is executed in advance on plural body tissues. Time periods from when application of ultrasonic energy is started until when the impedance of the ultrasonic transducer 922 becomes the predetermined value Th are acquired and an average of the acquired times is set as the aforementioned predetermined time on which the determination is made at step S16.

When it is determined that the predetermined time elapses from application of ultrasonic energy (step S16: YES), the control device 3 moves to step S10.

The above-described modification produces the same effect as that produced by the first embodiment and makes it possible to simplify the configuration by omitting the first and second sensors 32 and 35 and the first and second impedance calculators 362 and 363.

### Second Embodiment

A second embodiment of the present disclosure will be described.

In the following description, the same components as those of the first embodiment are denoted with the same reference numbers as those of the first embodiment and detailed description thereof will be omitted or simplified.

In the above-described medical treatment device 1 according to the first embodiment, the motor 11 and the load controller 364 are used as a configuration to increase the compressive load applied to subject parts when application of thermal energy is started, thereby automatically increasing the compressive load.

On the contrary, the medical treatment device according to the second embodiment employs a configuration in which an operator manually increases the compressive load applied to subject parts when application of thermal energy is started.

The configuration of the medical treatment device and joining control according to the second embodiment will be described.

Configuration of Medical Treatment Device

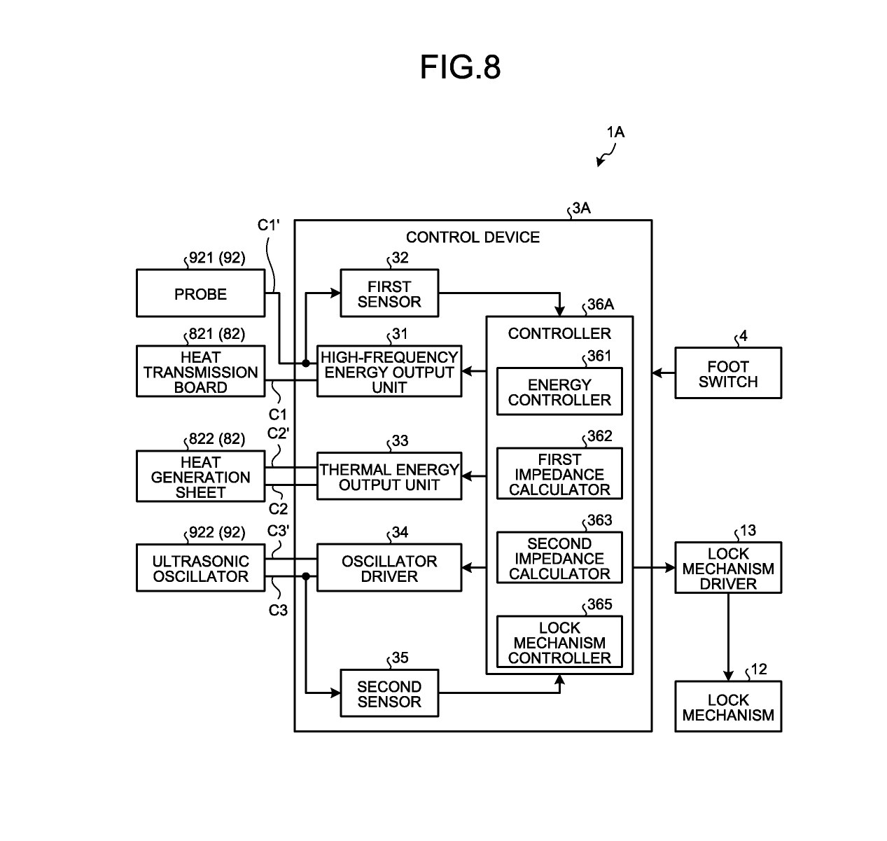

FIG. 8 is a block diagram illustrating a configuration of a medical treatment device 1A according to the second embodiment of the present disclosure.

In the medical treatment device 1A according to the second embodiment, as illustrated in FIG. 8, compared to the medical treatment device 1 (FIG. 1 and FIG. 2) described in the above-described first embodiment, the motor 11 and the load controller 364 are omitted. In the medical treatment device 1A, compared to the medical treatment device 1 described in the above-described first embodiment, a lock mechanism 12 and a lock mechanism driver 13 are added and part of the functions of the controller 36 is changed.

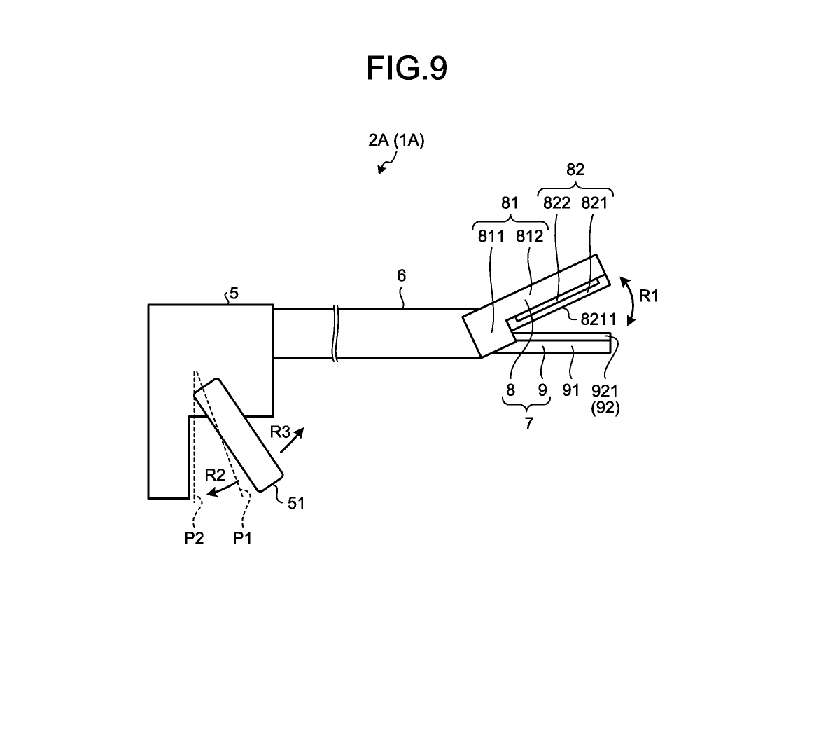

FIG. 9 is a diagram illustrating functions of the lock mechanism 12. Specifically, FIG. 9 is a diagram illustrating a treatment tool 2A according to the second embodiment.

The lock mechanism 12 is provided inside the handle 5 and switches the operation knob 51 between an allowed state and a regulated state.

Specifically, for the regulated sate, the lock mechanism 12 mechanically connects with (locks) the operation knob 51 or the open-close mechanism 10 to regulate movements of the operation knob 51 from First position P1 (FIG. 9) to Second position P2 (FIG. 9). In the allowed state, the lock mechanism 12 mechanically disconnects (unlocks) the operation knob 51 or the open-close mechanism 10 to allow the operation knob 51 to move.

First position P1 is the following position.

When the operation knob 51 moves from an initial position (the position of the operation knob 51 illustrated in FIG. 9) to First position P1, the first holding member 8 rotates in a direction in which the first holding member 8 gets close to the second holding member 9 and applies a relatively low compressive load (first compressive load (for example, approximately 0.2 MPa)) to subject parts that are clamped between the first holding member 8 and the second holding member 9. In other words, First position P1 is a position to apply the first compressive load to the subject parts.

Second position P2 is the following position.

When the operation knob 51 moves from First position P1 to Second position P2, the first holding member 8 rotates in a direction in which the first holding member 8 further gets close to the second holding member 9 and applies a second compressive load that is higher than the first compressive load to the subject parts that are clamped between the first holding member 8 and the second holding member 9. In other words, Second position P2 is a position to apply the second compressive load to the subject parts.

In the second embodiment, the lock mechanism 12 is biased by a biasing member, such as a spring, to be kept mechanically connected with (locks) the operation knob 51 or the open-close mechanism 10.

The lock mechanism driver 13 is provided inside the handle 5 and, under the control of a control device 3A (a controller 36A), causes the lock mechanism 12 to move against a biasing force of the biasing member, such as a spring, thereby switching the operation knob 51 from the regulated state to the allowed state.

As illustrated in FIG. 8, compared to the controller 36 (FIG. 2) described in the above-described first embodiment, the load controller 364 is omitted and a lock mechanism controller 365 is added.

The lock mechanism controller 365 drives the lock mechanism driver based on the impedance of the ultrasonic transducer 922 that is calculated by the second impedance calculator 363 and switches the operation knob 51 from the regulated state to the allowed state.

Joining Control

A joining control according to the second embodiment will be described.

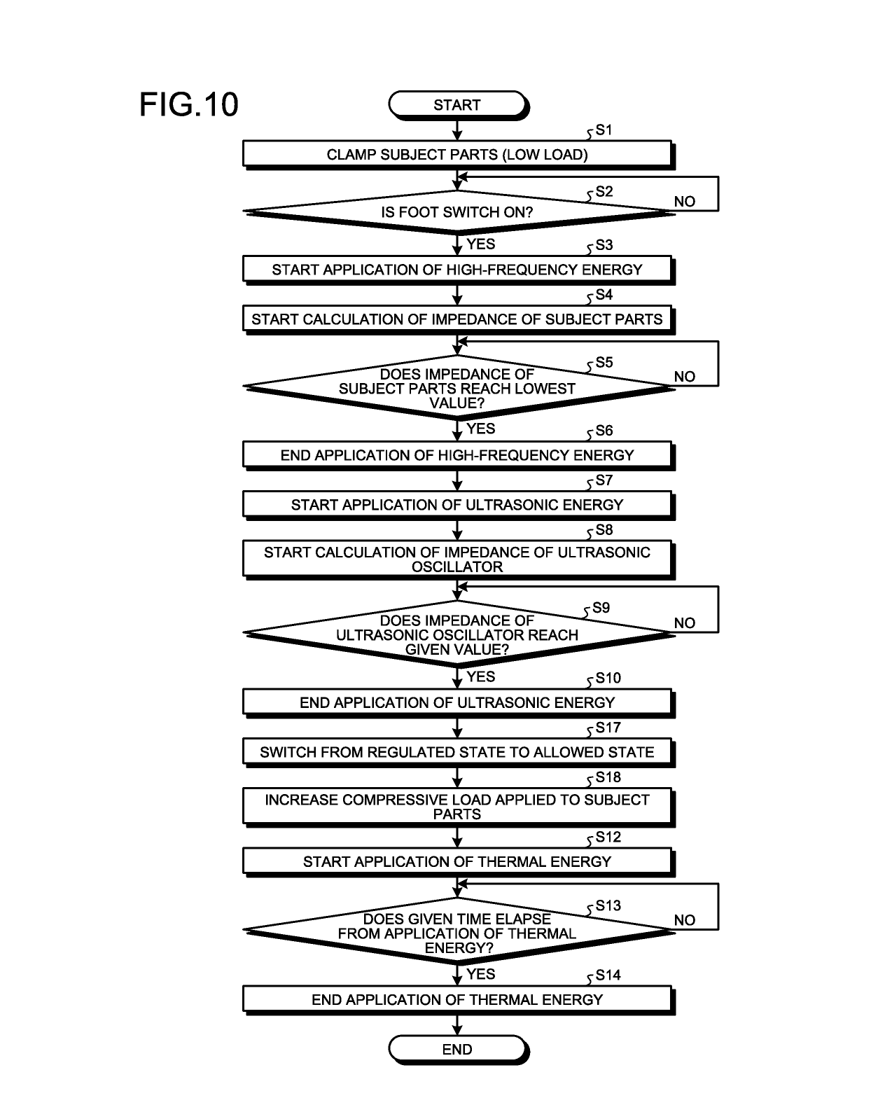

FIG. 10 is a flowchart illustrating the joining control performed by the control device 3A.

In the joining control according to the second embodiment, as illustrated in FIG. 10, compared to the joining control described in the above-described first embodiment (FIG. 3), step S11 relating to operations of the motor 11 is omitted and steps S17 and S18 are added.

As described above, in a state where the lock mechanism driver 13 is not driven, the lock mechanism 12 is biased by the biasing member, such as a spring, so as to mechanically connect with the operation knob 51 or the open-close mechanism 10 (the operation knob 51 is set in the regulated state). Thus, at step S1 in the second embodiment, the operator moves the operation knob 51 from the initial position to First position P1 to clamp the subject parts with the first and second holding members 8 and 9. In other words, the first compressive load is applied to the subject parts.

Step S17 is executed after step S10.

Specifically, at step S17, under the condition that the impedance of the ultrasonic transducer 922 is determined to have become the predetermined value Th at step S9 (step S9: YES), the lock mechanism controller 365 drives the lock mechanism driver 13 to switch the operation knob 51 from the regulated state to the allowed state.

After step S17, the operator moves the operation knob 51 from First position P1 to Second position P2 (step S18). In other words, the second compressive load higher than the first compressive load is applied to the subject parts.

After step S18, the control device 3A moves to step S12.

According to the above-explained second embodiment the following effect is produced in addition to the same effect as that of the above-described first embodiment.

The medical treatment device 1A according to the second embodiment has, as a configuration to increase the compressive load applied to subject parts when application of thermal energy is started, a configuration in which the lock mechanism 12 is used to allow the operator to increase the compressive load manually.

### Modification of Second Embodiment

In the above-described second embodiment, as in the modification of the first embodiment described above (FIG. 7), a configuration may be employed in which application of ultrasonic energy and thermal energy is started (the operation knob 51 is switched from the regulated state to the allowed state) when a predetermined time elapses.

In the above-described second embodiment, the medical treatment device 1A may be provided with a notifying unit that notifies that the operation knob 51 is switched from the regulated state to the allowed state.

As the notifying unit, a configuration to notify by turning on a light emitting diode (LED) or the like, a configuration to notify by displaying a message or the like, or a configuration to notify by making sound or the like, may be exemplified.

### Third Embodiment

A third embodiment of the present disclosure will be described.

A medical treatment device according to the third embodiment has the same configuration as that of the medical treatment device 1, with only a difference in energy applied to subject parts in First Period T1, with respect to the medical treatment device 1 according to the first embodiment. For this reason, in the following description, the same components as those of the first embodiment are denoted with the same reference numbers as those of the first embodiment and detailed description thereof will be omitted or simplified.

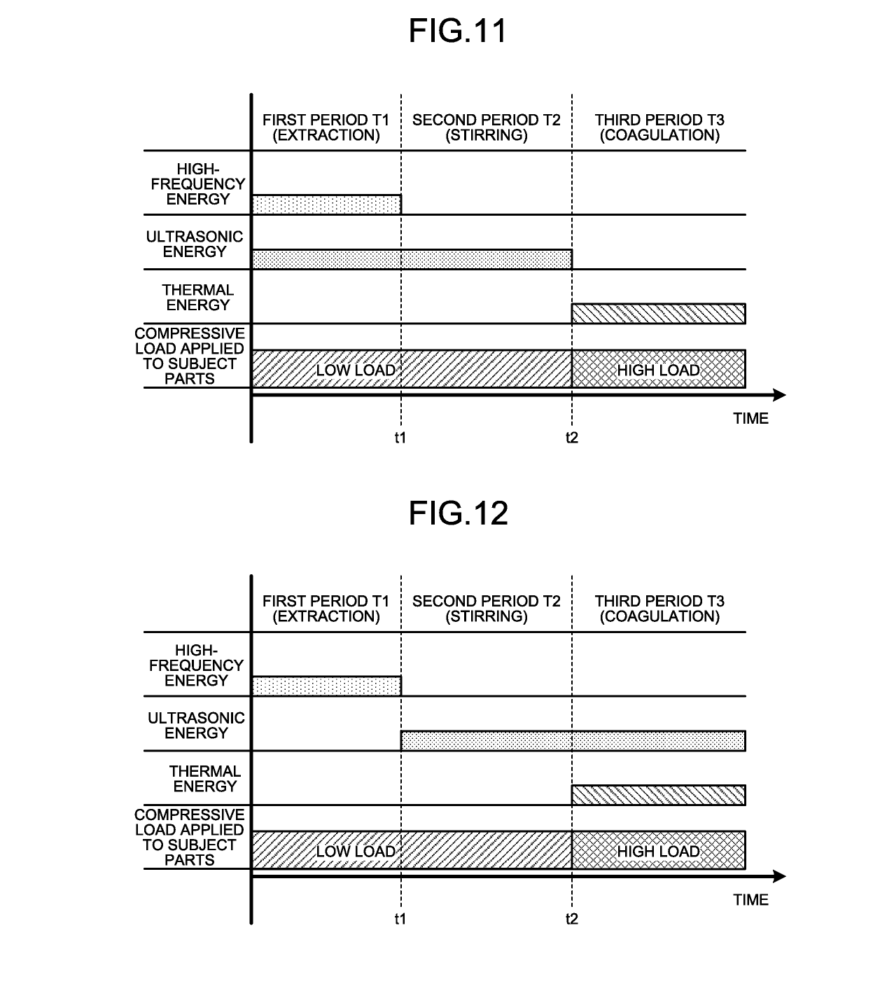

FIG. 11 is a time chart representing types of energy to be applied to the subject parts and compressive loads to be applied to the subject parts in First to Third Periods T1 to T3 in joining control according to the third embodiment of the present disclosure.

The medical treatment device 1 (the energy controller 361) according to the third embodiment applies ultrasonic energy in addition to high-frequency energy to subject parts in First Period T1 as illustrated in FIG. 11. The types of energy applied to the subject parts in Second and Third Periods T2 and T3 and the compressive loads applied to the subject parts in First to Third Periods T1 to T3 are the same as those in the first embodiment.

According to the above-explained third embodiment described above produces the following effect in addition to the same effect as that of the above-described first embodiment.

In the medical treatment device 1 according to the third embodiment, two types of energy, which are high-frequency energy and ultrasonic energy, are applied to the subject parts in First Period T1.

Accordingly, in addition to electric destruction of cell membrane by high-frequency energy, destruction of cell membrane by ultrasonic energy (ultrasonic oscillations) can be performed and destruction of the cell membrane is promoted so that the extracellular matrix can be extracted in a short time. Therefore, First Period T1 to extract the extracellular matrix becomes short, and, as a result, it is possible to shorten the time to treat the subject parts.

### Fourth Embodiment

A fourth embodiment of the present disclosure will be described.

A medical treatment device according to the fourth embodiment has the same configuration as that of the medical treatment device 1, with only a difference in energy applied to subject parts in Third Period T3, with respect to the medical treatment device 1 according to the first embodiment. For this reason, in the following description, the same components as those of the first embodiment are denoted with the same reference numbers as those of the first embodiment and detailed description thereof will be omitted or simplified.

FIG. 12 is a time chart representing types of energy to be applied to the subject parts and compressive loads to be applied to the subject parts in First to Third Periods T1 to T3 in joining control according to the fourth embodiment of the present disclosure.

The medical treatment device 1 (the energy controller 361) according to the fourth embodiment applies ultrasonic energy in addition to thermal energy to the subject parts in Third Period T3 as illustrated in FIG. 12. The types of energy applied to the subject parts in First and Second Periods T1 and T2 and the compressive loads applied to the subject parts in First to Third Periods T1 to T3 are the same as those in the first embodiment.

According to the above-explained fourth embodiment of the present disclosure, the following effect is produced in addition to the same effect as that of the above-described first embodiment.

In the medical treatment device 1 according to the fourth embodiment, two types of energy, which are thermal energy and ultrasonic energy, are applied to the subject parts in Third Period T3.

For this reason, by using the two types energy together, dehydration and coagulation are accelerated in tissue of the subject parts and thus it is possible to cause the extracellular matrix to coagulate in a short time. Therefore, Third Period T3 to cause the extracellular matrix to coagulate becomes shorter, and, as a result, it is possible to shorten the time to treat the subject parts.

### Fifth Embodiment

A fifth embodiment of the present disclosure will be described.

A medical treatment device according to the fifth embodiment has the same configuration as that of the medical treatment device 1, with only a difference in energy applied to subject parts in Third Period T3, with respect to the medical treatment device 1 according to the first embodiment. For this reason, in the following description, the same components as those of the first embodiment are denoted with the same reference numbers as those of the first embodiment and detailed description thereof will be omitted or simplified.

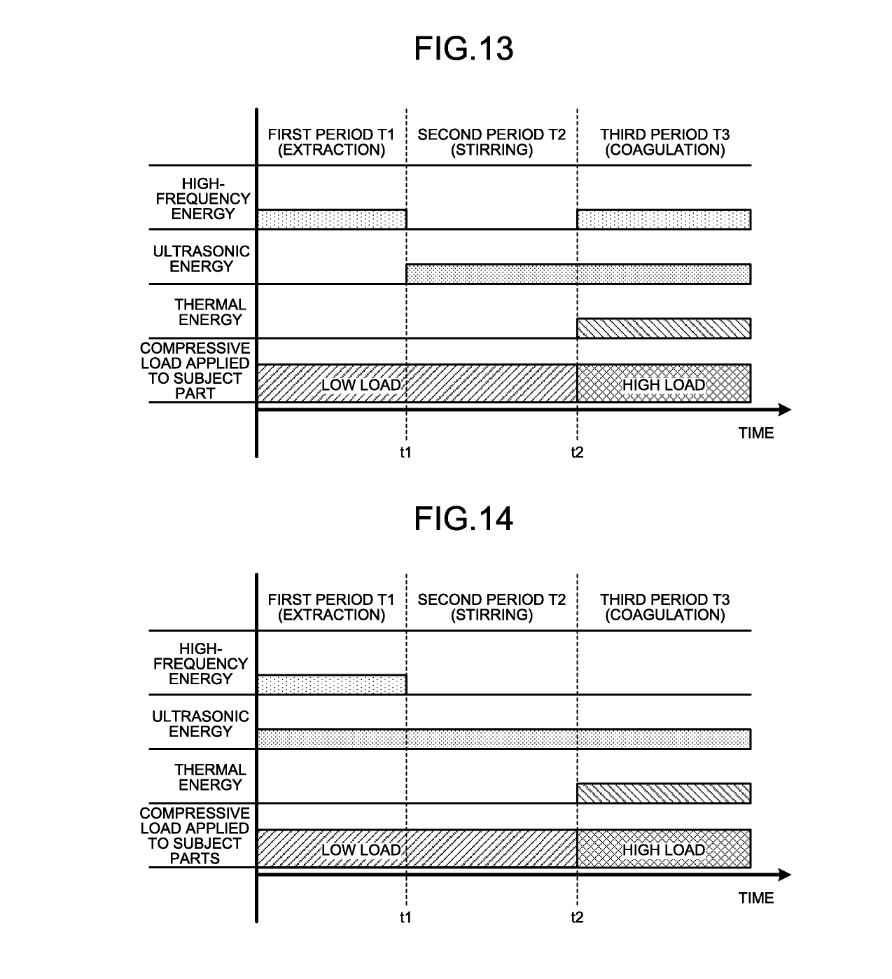

FIG. 13 is a time chart representing types of energy to be applied to the subject parts and compressive loads to be applied to the subject parts in First to Third Periods T1 to T3 in joining control according to the fourth embodiment of the present disclosure.

The medical treatment device 1 (the energy controller 361) according to the fifth embodiment applies high-frequency energy and ultrasonic energy in addition to thermal energy to the subject parts in Third Period T3 as illustrated in FIG. 13. The types of energy applied to the subject parts in First and Second Periods T1 and T2 and the compressive loads applied to the subject parts in First to Third Periods T1 to T3 are the same as those in the first embodiment.

According to the above-explained fifth embodiment the following effect is produced in addition to the same effect as that of the above-described first embodiment.

In the medical treatment device 1 according to the fifth embodiment, three types of energy, which are thermal energy, high-frequency energy and ultrasound energy are applied to the subject parts in Third Period T3.

Accordingly, by using the three types of energy together, dehydration and coagulation are accelerated in tissue of the subject parts and thus the extracellular matrix may coagulate in a short time. Therefore, Third Period T3 to cause the extracellular matrix to coagulate becomes shorter, and, as a result, it is possible to shorten the time to treat the subject parts.

### Sixth Embodiment

A sixth embodiment of the present disclosure will be described.

A medical treatment device according to the sixth embodiment has the same configuration as that of the medical treatment device 1, with only a difference in energy applied to subject parts in First and Third Periods T1 and T3, with respect to the medical treatment device 1 according to the first embodiment. For this reason, in the following description, the same components as those of the first embodiment are denoted with the same reference numbers as those of the first embodiment and detailed description thereof will be omitted or simplified.

FIG. 14 is a time chart representing types of energy to be applied to the subject parts and compressive loads to be applied to the subject parts in First to Third Periods T1 to T3 in joining control according to the sixth embodiment of the present disclosure.

The medical treatment device 1 (the energy controller 361) according to the sixth embodiment applies ultrasonic energy in addition to high-frequency energy to the subject parts in First Period T1 as in the above-described third embodiment as illustrated in FIG. 14. As illustrated in FIG. 14, in Third Period T3, the medical treatment device 1 (the energy controller 361) according to the sixth embodiment applies ultrasonic energy in addition to thermal energy to the subject parts as in the above-described fourth embodiment. The types of energy applied to the subject parts in Second Period T2, and the compressive loads applied to the subject parts in First to Third Periods T1 to T3 are the same as those in the first embodiment.

According to the above-explained sixth embodiment, the same effect as those of the first, third and fourth embodiments are produced.

### Seventh Embodiment

A seventh embodiment of the present disclosure will be described.

A medical treatment device according to the seventh embodiment has the same configuration as that of the medical treatment device 1 with only a difference in energy applied to subject parts in First and Third Periods T1 and T3, with respect to the medical treatment device 1 according to the first embodiment. For this reason, in the following description, the same components as those of the first embodiment are denoted with the same reference numbers as those of the first embodiment and detailed description thereof will be omitted or simplified.

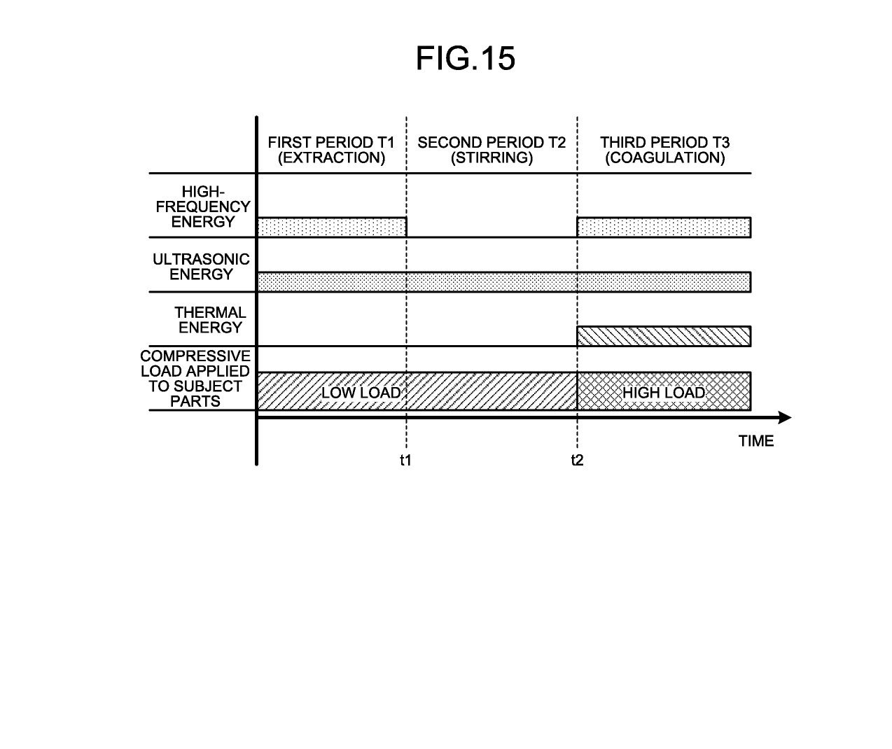

FIG. 15 is a time chart representing types of energy to be applied to the subject parts and compressive loads to be applied to the subject parts in First to Third Periods T1 to T3 in joining control according to the seventh embodiment of the present disclosure.

As in the above-described third embodiment, the medical treatment device 1 (the energy controller 361) according to the seventh embodiment applies ultrasonic energy in addition to high-frequency energy to the subject parts in First Period T1 as illustrated in FIG. 15. Furthermore, as in the above-described fifth embodiment, the medical treatment device 1 (the energy controller 361) according to the seventh embodiment applies high-frequency energy and ultrasonic energy in addition to thermal energy to the subject parts in Third Period T3 as illustrated in FIG. 15. The types of energy applied to the subject part in Second Periods T2, and the compressive loads applied to the subject parts in First to Third Periods T1 to T3 are the same as those in the first embodiment.

According to the above-described seventh embodiment, the same effect as those of the first, third and fifth embodiments are produced.

### Modifications of Third to Seventh Embodiments

The configurations described in the above-described third to seventh embodiments, and the configuration described in the above-described modification of the first embodiment, the configuration described in the above-described second embodiment, or the configuration described in the above-described modification of the second embodiment may be combined as appropriate.

### Other Embodiments

Modes for carrying out the present disclosure have been described so far; however, the disclosure should not be limited only by the above-described first to seventh embodiments and the modifications thereof.

In the first to seventh embodiments and modifications thereof, the first energy application unit 82 is provided in the first holding member 8 and the second energy application unit 92 is provided in the second holding member 9; however, the configuration is not limited to this, and any configuration in which an energy application unit that applies each of high-frequency energy, ultrasonic energy and thermal energy to subject parts is provided to only one of the first and second holding members 8 and 9 may be employed as long as the configuration enables application of the energies. Alternatively, a configuration in which each of the energy application units is provided to both the first and second holding members 8 and 9 may be employed. For example, the heat generation sheet 822 and the heat transmission board 821 may be formed on the probe 921.

In the first to seventh embodiments and the modifications thereof, the heat generation sheet 822 is used as a configuration to apply thermal energy to subject parts; however, the configuration is not limited to this. A configuration may be employed, for example, in which plural heat generation chips are provided on the heat transmission board 821, and the plural heat generation chips are energized to transmit the heat of the plural heat generation chips to the subject parts via the heat transmission board 821 (as for the technology, for example, refer to Japanese Laid-open Patent Publication No. 2013-106909).

In the first to seventh embodiments and the modifications thereof, the timing of starting Second and Third Periods T2 and T3 is adjusted based on the impedances of the subject parts and the ultrasonic transducer 922 and time; however, timing adjustment is not limited thereto. For example, the aforementioned timing may be adjusted based on a physical property, such as the hardness, thickness or the temperature of the subject parts.

In the above-described first to seventh embodiments, Second Period T2 is started when the impedance of the subject parts becomes the lowest value VL; however, the starting is not limited to this. Second Period T2 may be started at any timing as long as the timing is after Time t1 at which the impedance of the subject parts becomes the lowest value VL (for example, from Time t1 until Time t1′ (FIG. 4) at which the impedance recovers to the initial value VI (FIG. 4) at the time point when application of high-frequency energy is started).

The flow of joining control is not limited to the process order in the flowcharts (FIG. 3, FIG. 7 and FIG. 10) described in the above-described first to seventh embodiments and modifications thereof and may be changed as long as no inconsistency is caused.

The medical treatment device, the operation method of a medical treatment device and the treatment method according to the present disclosure produces an effect that it is possible to increase the strength of joining between subject parts.

What is claimed is:

1. A medical treatment device comprising:
a pair of holding members with which a subject part to be joined in a body tissue is clampable;
an energy application unit provided on at least one of holding members of the pair of holding members, so as to contact the subject part when the subject part is clamped by the pair of holding members, thereby to apply energy to the subject part; and
a processor comprising hardware, the processor being configured to control the energy to be applied from the energy application unit to the subject part, thereby to treat the subject part, wherein
in a period from when application of energy to the subject part is started until when treating the subject part is completed, the processor is further configured to cause the energy application unit to:
apply at least high-frequency energy to the subject part in a first period;
apply only ultrasonic energy to the subject part in a second period after the first period; and
apply at least thermal energy to the subject part in a third period after the second period.

2. The medical treatment device according to claim 1, wherein the processor is further configured to cause the energy application unit to apply ultrasonic energy to the subject part in the first period.

3. The medical treatment device according to claim 1, wherein the processor is further configured to cause the energy application unit to apply ultrasonic energy to the subject part in the third period.

4. The medical treatment device according to claim 1, wherein the processor is further configured to cause the energy application unit to apply high-frequency energy and ultrasonic energy to the subject part in the third period.

5. The medical treatment device according to claim 2, wherein the processor is further configured to cause the energy application unit to apply ultrasonic energy to the subject part in the third period.

6. The medical treatment device according to claim 2, wherein the processor is further configured to cause the energy application unit to apply high-frequency energy and ultrasonic energy to the subject part in the third period.

7. The medical treatment device according to claim 1, wherein
the processor is further configured to
calculate an impedance of the subject part during application of high-frequency energy to the subject part, and
start the second period after the impedance of the subject part calculated by the first impedance calculator becomes a lowest value.

8. The medical treatment device according to claim 1, wherein
the energy application unit includes an ultrasonic transducer that allows apply ultrasonic energy to be applied to the subject part, and
the processor is further configured to
calculate an impedance of the ultrasonic transducer during application of ultrasonic energy to the subject part, and
start the third period when the impedance of the ultrasonic transducer calculated by the second impedance calculator becomes a value set in advance.

9. The medical treatment device according to claim 1, wherein the processor is further configured to
control the pair of holding members so as to change a compressive load applied to the subject part from the pair of holding members, when the subject part is clamped by the pair of holding members, and
set the compressive load at different loads in the first period, the second period and the third period, respectively.

10. The medical treatment device according to claim 9, wherein the processor is further configured to set the compressive load higher in the third period than in the first period and the second period.

11. The medical treatment device according to claim 1,
wherein the pair of holding members are configured as relatively movable to a first position where a first compressive load is applied to the subject part and a second position where a second compressive load higher than the first compressive load is applied to the subject part, and
wherein the medical treatment device further comprises:
a lock mechanism that is switchable between an allowed state where relative movement of the pair of holding members from the first position to the second position is allowed and a regulated sate where the relative movement from the first position to the second position is regulated; and
wherein the processor is further configured to
set the lock mechanism into the regulated state in the first period and the second period, and
set the lock mechanism into the allowed state in the third period.

12. An operation method of a medical treatment device, the method comprising:
applying, after subject part to be joined in a body tissue is clamped by a pair of holding members, at least high-frequency energy from at least one of holding members of the pair of holding members to the subject part in a first period,
applying only ultrasonic energy from at least one of holding members of the pair of holding members to the subject part in a second period after the first period, and
applying at least thermal energy from at least one of holding members of the pair of holding members to the subject part in a third period after the second period.

13. A treatment method comprising:
clamping a subject part to be joined in a body tissue with a pair of holding members;
applying at least high-frequency energy from at least one of holding members of the pair of holding members to the subject part in a first period,
applying only ultrasonic energy from at least one of holding members of the pair of holding members to the subject part in a second period after the first period, and
applying at least thermal energy from at least one of holding members of the pair of holding members to the subject part in a third period after the second period.
